(19) United States
(12) Patent Application Publication
(10) Pub. No.: US 2012/0024089 A1
(43) Pub. Date: Feb. 2, 2012

(54) METHODS OF TEACHING BONDING LOCATIONS AND INSPECTING WIRE LOOPS ON A WIRE BONDING MACHINE, AND APPARATUSES FOR PERFORMING THE SAME

(75) Inventors: Jeremiah Couey, Elkins Park, PA (US); Michael T. Deley, Warrington, PA (US); Shawn Sarbacker, North Wales, PA (US); Matthew Odhner, Bryn Athyn, PA (US)

(73) Assignee: KULICKE AND SOFFA INDUSTRIES, INC., Fort Washington, PA (US)

(21) Appl. No.: 12/920,105

(22) PCT Filed: Feb. 29, 2008
(86) PCT No.: PCT/US2008/055407

§ 371 Date: Aug. 28, 2010

(51) Int. Cl. G01N 33/00; B23K 37/00; B23K 20/00

(52) U.S. Cl. 73/865.8; 228/102; 228/4.5

(57) ABSTRACT

A method of teaching bonding locations of a semiconductor device on a wire bonding machine is provided. The method includes (1) providing the wire bonding machine with position data for (a) bonding locations of a first component of the semiconductor device, and (b) bonding locations of a second component of the semiconductor device; and (2) teaching the bonding locations of the first component of the semiconductor device and the second component of the semiconductor device using a pattern recognition system of the wire bonding machine to obtain more accurate position data for at least a portion of the bonding locations of the first component and the second component. The teaching step is conducted by teaching the bonding locations in the order in which they are configured to be wire bonded on the wire bonding machine.

### CROSS REFERENCE

This application claims the benefit of International Application No. PCT/US2008/055407 filed Feb. 29, 2008, the contents of which is incorporated herein by reference.

### FIELD OF THE INVENTION

The present invention relates to the operation of a wire bonding machine, and more particularly, to improved methods of teaching bonding locations and inspecting wire loops on a wire bonding machine.

### BACKGROUND OF THE INVENTION

U.S. Pat. Nos. 5,119,435; 5,119,436; 5,125,036; 5,600,733; and 6,869,869 relate to wire bonding systems and associated methods of operating the wire bonding systems, and are hereby incorporated by reference in their entirety.

In the processing and packaging of semiconductor devices, wire bonding continues to be the primary method of providing electrical interconnection between two locations within a package (e.g., between a die pad of a semiconductor die and a lead of a leadframe). More specifically, using a wire bonder (also known as a wire bonding machine), wire loops are formed between respective locations to be electrically interconnected. FIG. 1A illustrates exemplary components of a portion of a wire bonding machine including optics assembly 18 (including camera portion 18a), transducer 14 (e.g., an ultrasonic transducer), bonding tool 16 (e.g., a capillary wire bonding tool, a wedge bonding tool, etc.), device clamp 12, and heat block 10. As is known to those skilled in the art, elements 14, 18, and 18a (amongst other non-illustrated components) are part of what is known as the “bond head” of the wire bonding machine, where the bond head moves about during wire bonding (and other operations such as teaching) using an xy table. As is known to those skilled in the art, a device to be wire bonded (e.g., a semiconductor die positioned on a substrate/leadframe) is positioned on heat block 10, and then secured by device clamp 12. After the device is secured in place, the wire bonding operation is performed using bonding tool 16 which bonds wire loops between bonding locations of the device to be wire bonded. The device to be wire bonded is accessible through aperture 12a of device clamp 12.

A portion of an exemplary semiconductor device is shown in a cut away side view in FIG. 1B. The device includes semiconductor die 102 supported by substrate 100 (e.g., a leadframe 100). Wire loops 104 have been bonded between (1) bonding locations on semiconductor die 102 (i.e., die pads 102a, 102i, etc.) and (2) bonding locations on leadframe 100 (i.e., leads 100a, 100i, etc.). FIG. 2 is a top view of a device similar to that shown in FIG. 1B. As shown in FIG. 2, leadframe 100 includes leads 100a, 100b, 100c, 100d, 100e, 100f, 100g, 100h, 100i, 100j, 100k, and 100l. Leadframe 100 also includes leadframe eyepoints 100a1 and 100a2. Semiconductor die 102 includes die pads 102a, 102b, 102c, 102d, 102e, 102f, 102g, 102h, 102i, 102j, 102k, and 102l. Semiconductor die 102 also includes eyepoints 102a1 and 102a2. As shown in FIG. 2, wire loops 104 are extended between corresponding ones of the die pads of semiconductor die 102 and the leads of leadframe 100. For example, a wire loop 104 provides electrical interconnection between die pad 102a and lead 100a. Likewise, another wire loop 104 provides electrical interconnection between die pad 102b and lead 100b, and so on.

Teaching operations using vision systems (e.g., Pattern Recognition Systems or PRS) are often utilized in connection with wire bonding operations. For example, before a wire bonding operation is performed on a batch of semiconductor devices (e.g., devices such as a semiconductor die mounted on a leadframe), it is typically desired to “teach” an eyepoint (or multiple eyepoints) of a sample device. Further, bonding locations of a sample device (e.g., die pads of a semiconductor die) may also be taught. By “teaching” the sample device, certain physical data related to the sample device is stored (e.g., in the memory of a wire bonding machine). This physical data is used as a reference during processing of the batch of devices, for example, to ensure proper positioning or alignment of each of the batch of semiconductor devices to be processed (e.g., to be wire bonded).

Such a teaching operation on a wire bonding machine may be the first time that data related to the position of the bonding locations and eyepoints of the sample device is provided to the memory of the wire bonding machine. Consider, for example, a situation where a sample device for which no position data is available is to be wire bonded. Such a device may be taught using the vision system of the wire bonding machine. In certain applications, however, the teaching operation on the wire bonding machine may be a confirmation of the position data previously provided to the wire bonding machine (e.g., offline using CAD data or the like).

Certain conventional techniques (e.g., algorithms that select, scan, and store the taught information) are used in conjunction with a vision system to perform the teaching operations. In many conventional systems, the eyepoints/bonding locations of a substrate/leadframe are taught independently of the eyepoints/bonding locations of the semiconductor die mounted on the substrate. For example, FIG. 3 illustrates an exemplary conventional sequence for teaching the eyepoints/bonding locations of substrate 100, while FIG. 4 illustrates an exemplary conventional sequence for teaching the eyepoints/bonding locations of semiconductor die 102. Referring specifically to FIG. 3, eyepoints 100a1 and 100a2 are taught in a first step (illustrated by the sequential labels “a” and “b”). Then, the leads are taught in a sequential order. More specifically, lead 100a is taught (as indicated by the label “1”), then lead 100b is taught (as indicated by the label “2”), then lead 100c is taught (as indicated by the label “3”), and so on, until lead 100l is taught (as indicated by the label “12”).

Referring specifically to FIG. 4, eyepoints 102a1 and 102a2 are taught in a first step (illustrated by the sequential labels “a” and “b”). Then, the die pads of semiconductor die 102 are taught in a sequential order. More specifically, die pad 102a is taught (as indicated by the label “1”), then die pad 102b is taught (as indicated by the label “2”), then die pad 102c is taught (as indicated by the label “3”), and so on, until then die pad 102l is taught (as indicated by the label “12”).

FIG. 5 illustrates an alternative approach useful for illustrating an option on a Model 1488 plus Automatic Gold Ball Bonder previously sold by Kulicke and Soffa Industries, Inc. In order to save time (and to provide an acceptable level of accuracy), bonding locations to be interconnected are taught in rows. Referring to FIG. 5, row “A” includes die pads 102a, 102b, and 102c, as well as leads 100a, 100b, and 100c. In the sequence shown in FIG. 5, die pad 102a is taught (as indicated by the label “1”). Then lead 100a is taught (as indicated by the label “2”). Thus, initially the two bonding locations at one end of row A are taught. Then, the vision system proceeds to the other end of the row and teaches die pad 102c (as indicated by the label “3”) followed by teaching lead 100c (as indicated by the label “4”). Thus, at this point in the teach process, each end of row A has been taught. Thereafter, the system is configured to teach the bonding locations in between the two ends of the row, proceeding from a die pad to the corresponding lead, then to the next corresponding die pad, then to the next corresponding lead, and so on. As shown in FIG. 5, die pad 102b (as indicated by the label “5”) is now taught, followed by the teaching of lead 100b (as indicated by the label “6”). If additional bonding locations existed in row A, they would be taught by proceeding from a die pad to the corresponding lead, then to the next corresponding die pad, then to the next corresponding lead, and so on. This is illustrated by the zig-zag dotted line extending from lead 100b.

The conventional teaching processes described above (as well as other conventional teaching processes) may have provided acceptable results when the spacing (and size) of bonding locations is relatively large, and/or when the spacing is relatively uniform; however, the conventional teaching processes are subject to various error sources that result in an undesirable level of measurement variance. The conventional techniques tend to be even more problematic as the spacing (and the uniformity of the spacing, and the size of the bonding locations) of bonding locations continues to shrink.

Thus, it would be desirable to provide improved methods of teaching bonding locations using a wire bonding machine.

### SUMMARY OF THE INVENTION

According to an exemplary embodiment of the present invention, a method of teaching bonding locations of a semiconductor device on a wire bonding machine is provided. The method includes (1) providing the wire bonding machine with position data for (a) bonding locations of a first component of the semiconductor device, and (b) bonding locations of a second component of the semiconductor device; and (2) teaching the bonding locations of the first component of the semiconductor device and the second component of the semiconductor device using a pattern recognition system of the wire bonding machine to obtain more accurate position data for at least a portion of the bonding locations of the first component and the second component. The teaching step is conducted by teaching the bonding locations in the order in which they are configured to be wire bonded on the wire bonding machine.

According to another exemplary embodiment of the present invention, a method of teaching bonding locations of a semiconductor device on a wire bonding machine is provided. The method includes (1) teaching a plurality of bonding locations of a first component of the semiconductor device and a second component of the semiconductor device using a pattern recognition system of the wire bonding machine, the teaching step being conducted by teaching the bonding locations in the order in which they are configured to be wire bonded on the wire bonding machine, the teaching step including repeating the teaching of the bonding locations a predetermined number of times to obtain position data for each of the bonding locations for each of the repeated steps of teaching; and (2) arithmetically deriving more accurate position data for the bonding locations by utilizing position data obtained from the repeated teaching of the bonding locations.

According to another exemplary embodiment of the present invention, a method of inspecting wire loops of a semiconductor device on a wire bonding machine is provided. The method includes (1) providing a semiconductor device including a plurality of wire loops, each of the wire loops providing electrical interconnection between a first bonding location of the semiconductor device and a second bonding location of the semiconductor device; and (2) inspecting predetermined portions of the wire loops using a pattern recognition system of the wire bonding machine, the inspecting step being conducted by moving a portion of the pattern recognition system to scan the predetermined portions of the wire loops at the respective bonding locations in the order in which they were wire bonded on the wire bonding machine.

The methods of the present invention may also be embodied as an apparatus (e.g., as part of the intelligence of a wire bonding machine), or as computer program instructions on a computer readable carrier (e.g., a computer readable carrier used in connection with a wire bonding machine).

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1A is a block diagram perspective view of components of a wire bonding machine used in accordance with an exemplary embodiment of the present invention;

FIG. 1B is a cut-away side view of a semiconductor device including wire loops providing electrical interconnection between a leadframe and a semiconductor die;

FIG. 2 is a block diagram top view of a semiconductor device including wire loops providing electrical interconnection between a leadframe and a semiconductor die;

FIG. 3 is a block diagram top view of a leadframe illustrating a conventional technique for teaching leads of the leadframe;

FIG. 4 is a block diagram top view of a semiconductor die illustrating a conventional technique for teaching die pads of the semiconductor die;

FIG. 5 is a block diagram top view of a semiconductor device illustrating a conventional technique for teaching bonding locations of the semiconductor device;

FIG. 6 is a block diagram top view of a semiconductor device illustrating a technique for teaching bonding locations of the semiconductor device in accordance with an exemplary embodiment of the present invention;

FIG. 7 is a block diagram top view of a semiconductor device illustrating a technique for teaching bonding locations of the semiconductor device in accordance with another exemplary embodiment of the present invention;

FIG. 8 is a block diagram top view of a semiconductor device illustrating a technique for teaching bonding locations of the semiconductor device in accordance with yet another exemplary embodiment of the present invention;

FIG. 9 is a block diagram top view of a device clamp of a wire bonding machine defining an aperture through which a plurality of semiconductor devices to be wire bonded are accessible, the block diagram illustrating a technique for teaching bonding locations of one of the semiconductor devices in accordance with an exemplary embodiment of the present invention;

FIG. 10 is a block diagram top view of a device clamp of a wire bonding machine defining an aperture through which a plurality of semiconductor devices to be wire bonded are accessible, the block diagram illustrating a technique for teaching bonding locations of one of the semiconductor devices in accordance with another exemplary embodiment of the present invention;

FIG. 11 is a block diagram top view of a device clamp of a wire bonding machine defining an aperture through which a plurality of semiconductor devices to be wire bonded are accessible, the block diagram illustrating a technique for teaching bonding locations of one of the semiconductor devices in accordance with yet another exemplary embodiment of the present invention;

FIG. 12 is a block diagram top view of a device clamp of a wire bonding machine defining an aperture through which a plurality of semiconductor devices to be wire bonded are accessible, the block diagram illustrating a technique for teaching bonding locations of each of the semiconductor devices in accordance with an exemplary embodiment of the present invention;

FIG. 13A is a block diagram top view of a device clamp of a wire bonding machine defining an aperture through which another plurality of semiconductor devices to be wire bonded are accessible, the block diagram illustrating a technique for teaching bonding locations of one of the semiconductor devices in accordance with another exemplary embodiment of the present invention;

FIG. 13B is a block diagram top view of a device clamp of a wire bonding machine defining a aperture through which another plurality of semiconductor devices to be wire bonded are accessible, the block diagram illustrating a technique for teaching bonding locations of each of the semiconductor devices in accordance with yet another exemplary embodiment of the present invention;

FIG. 14 is a diagram illustrating a technique of teaching a bonding location where more accurate position data is arithmetically derived for the bonding location in accordance with yet another exemplary embodiment of the present invention;

FIG. 15 is a block diagram top view of a semiconductor device illustrating a technique for inspecting portions of wire loops in accordance with an exemplary embodiment of the present invention;

FIG. 16 is a block diagram top view of a semiconductor device illustrating a technique for inspecting portions of wire loops in accordance with another exemplary embodiment of the present invention;

FIG. 17 is a diagram illustrating a technique of inspecting a portion of a wire loop by arithmetically deriving more accurate inspection data for the portion of the wire loop in accordance with yet another exemplary embodiment of the present invention;

FIG. 18 is a flow diagram illustrating a method of teaching bonding locations of a semiconductor device and additional steps in accordance with an exemplary embodiment of the present invention; and

FIG. 19 is a block diagram of a portion of the intelligence of a wire bonding machine in accordance with an exemplary embodiment of the present invention.

### DETAILED DESCRIPTION OF THE INVENTION

As used herein, the term “components” of a semiconductor device refers any two portions of a semiconductor device that include bonding locations to be connected using wire loops. For example, a first component of a semiconductor device may be a substrate including bonding locations (e.g., a leadframe including leads), and a second component of the semiconductor device may be semiconductor die mounted on the substrate. In such a configuration the two components (e.g., leads on the leadframe, and die pads of the semiconductor die) may be connected using wire loops. In another example, each of the first component and the second component may be semiconductor dice, where die pads of each of the semiconductor dice are to be connected in die-to-die bonding (i.e., wire loops provide interconnection between die pads of the two semiconductor dice). Of course, other components are contemplated which may include bonding locations to be interconnected using wire loops.

As used herein, the term “wire bonding machine” is intended to broadly refer to any of a class of machines which may be used to bond wire portions. For example, such a machine may be configured to form wire loops. In other example, the machine may be configured to form conductive bumps (e.g., stud bumps or the like formed using wire). Of course, a single machine may be configured to form wire loops, conductive bumps, etc. Likewise, as will be understood by those skilled in the art, many of the aspects of the present invention are applicable to teaching and inspection of conductive bumps of semiconductor devices.

As is known to those skilled in the art, the teaching of eyepoints and bonding locations on a wire bonding machine is a process by which the eyepoints and bonding locations are scanned into images. The scanned images may then be analyzed (e.g., by the PRS) to determine information about the eyepoint/bonding location (e.g., information such as the relative position of the eyepoint/bonding location).

Various aspects of the present invention relate to teaching processes/techniques/algorithms. Of course, the use of the expression “teaching” is intended to cover any of a number of teaching operations including initial teaching operations, re-teaching operations, etc.

As provided above, teaching of eyepoints and bonding locations is subject to several error sources that results in measurement variance. Exemplary error sources may include: xy table following error, xy tablemapping error, servo dither error, machine vibration error, hysteresis error, thermal drift error, optical resolution error, and many other potential error sources. Therefore, the process of locating and teaching eyepoints and bonding locations introduces positional uncertainty of the actual position of the eyepoints and bonding locations. During the teach process these measurement uncertainties can lead to systematic errors to be taught into the bonding locations relative to the eyepoint locations, leading to wirebond placement accuracy error when the taught bonding locations are used in connection with the wire bonding operation.

As is known to those skilled in the art, it is often desirable to inspect wire loops (or portions of wire loops), for example, to determine if portions of the wire loop (e.g., a first ball bond portion of the wire loop) have been bonded accurately to a given bonding location. Such inspection and metrology processes are often referred to as post bond inspection (PBI) by those skilled in the art. For placement accuracy, PBI operations use the wire bonder vision system (e.g., a pattern recognition system or PRS) to find a portion of the bonded wire and to determine the position of the bonded wire portion relative to the previously taught bonding locations (or relative to the bonding location by finding the bonding location at the same time). As in the teach process, finding the bonded wire is subject to various error sources which introduces positional uncertainty of the actual position of the bonded wire relative to the taught bonding location position. Further, conventional xy table paths during PBI operations also have a significant influence on how the error sources contribute to the measurement uncertainty.

According to various exemplary embodiments of the present invention, there is a substantial reduction in the teach process error source contribution by configuring the xy table path (including the direction and distance) traveled during the teach process to be the same as the xy table path traveled during the wire bonding process. By conducting the teach process in this manner, accuracy of the teaching process is improved because of the omission of various error contributions related to the difference between the xy table path traveled during (1) the teaching operation, and (2) the wire bonding operation. In certain exemplary embodiments of the present invention, the teach process is automatically repeated multiple times so that multiple images of each bonding location are obtained and may be sampled (or otherwise mathematically manipulated) to obtain more accurate position data for the bonding locations, thereby reducing the potential measurement errors.

Furthermore, certain exemplary inventive techniques may be utilized in connection with a PBI process to reduce the error source contribution to the inspection process during the PBI process to be the same as the xy table path that was used during the actual wire bonding process. Furthermore, the PBI operation may be repeated and sampled (or otherwise mathematically manipulated) to further reduce the error contributions.

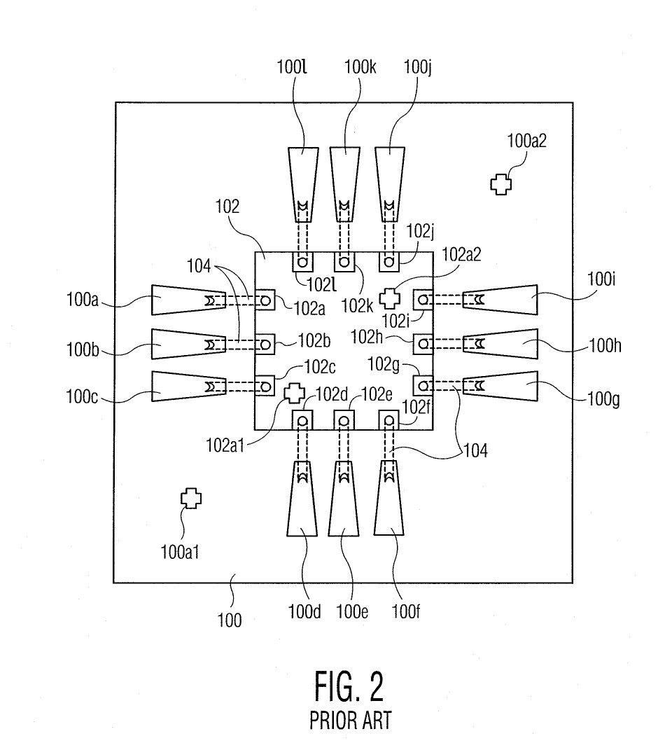

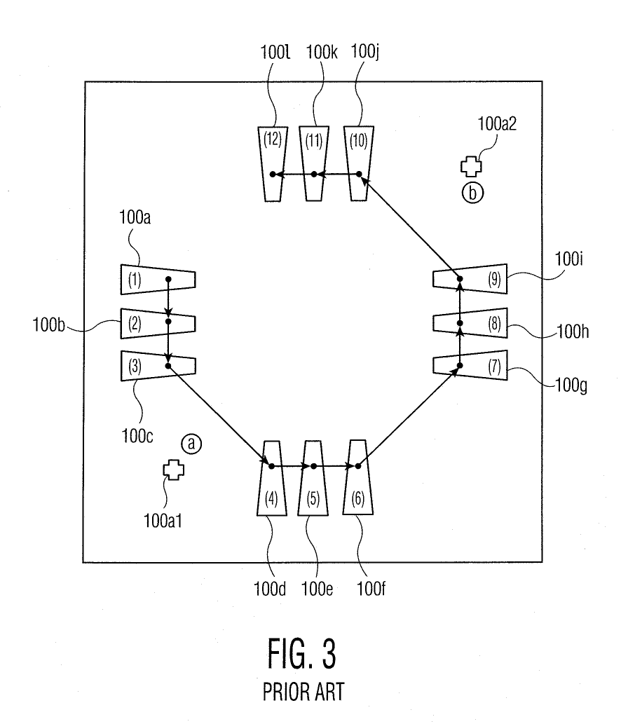

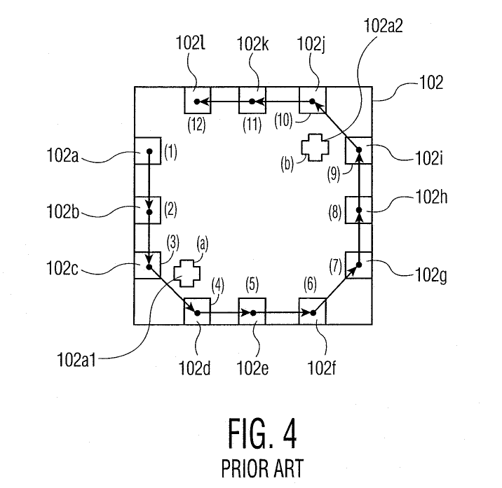

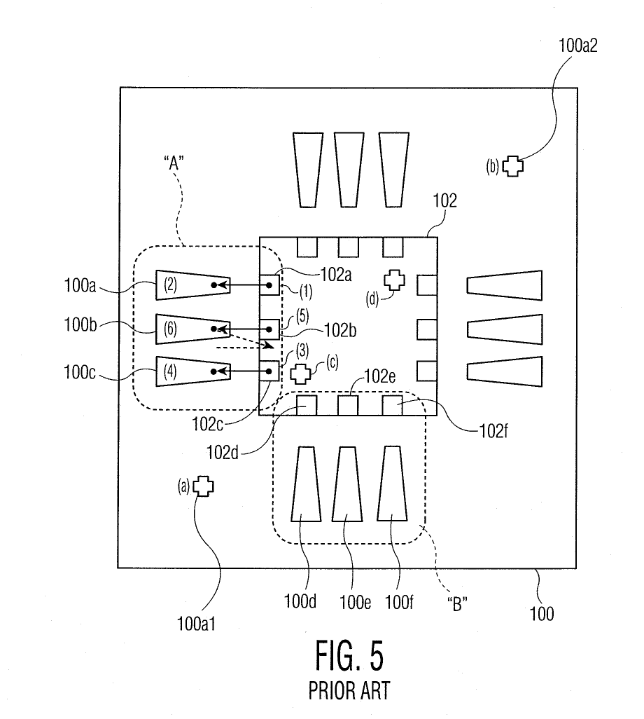

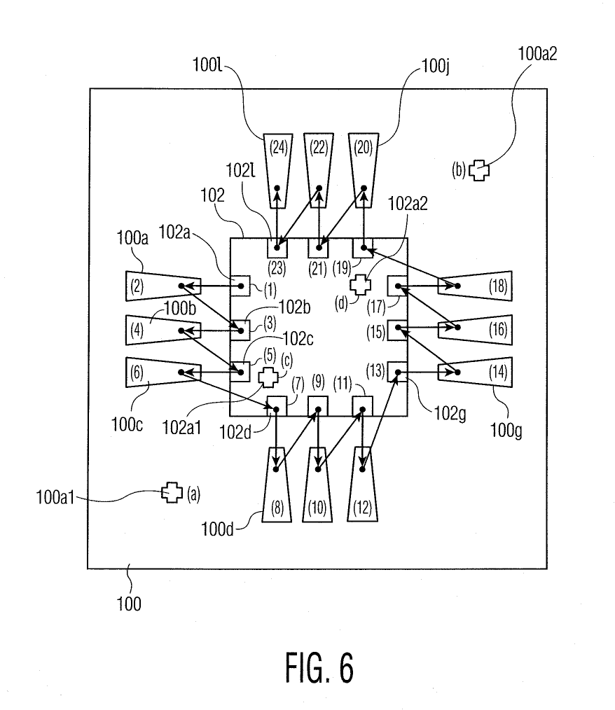

FIG. 6 illustrates a semiconductor device including semiconductor die 102 suppported by leadframe 100. The illustrated portions of semiconductor die 102 and leadframe 100 are the same as in FIG. 2, and as such, not all of the portions are labelled in FIG. 6 and subsequent figures. For example, only die pads 102a, 102b, 102c, 102d, 102g, and 102l are labelled in FIG. 6; however it is clear that the remaining die pads illustrated are the same as those shown in FIG. 2.

FIG. 6 illustrates a method of teaching eyepoints and bonding locations (in FIG. 6 the bonding locations are die pads and leads) in accordance with an exemplary embodiment of the present invention. The eyepoints are first taught in a predetermined order (e.g., in accordance with exemplary embodiments of the present invention, the predetermined order for teaching the eyepoints is the same order in which the eyepoints will be taught/scanned during a wire bonding operation). In the example shown in FIG. 6, the order is from a-d, that is, leadframe eyepoint 100a1 is taught first (as indicated by the label “a”), then leadframe eyepoint 100a2 is taught second (as indicated by the label “b”), then semiconductor die eyepoint 102a2 is taught third (as indicated by the label “c”), and then semiconductor die eyepoint 102a2 is taught fourth (as indicated by the label “d”). Of course, this order is exemplary in nature and the order in which the eyepoints are taught may vary.

After the eyepoints are taught/scanned, the bonding locations are to be taught. In the example shown in FIG. 6, the bonding locations are taught in the order in which they are to be wirebonded (in the order of “1” through “24” as labelled in FIG. 6). Thus, for the illustrated semiconductor device (including die 102 and leadframe 100), the wire bonder program is configured to bond wires starting with a wire from die pad 102a to lead 100a. More specifically, the bond program is configured to form a first bond of a wire loop at die pad 102a, and then is configured to extend a length of wire to the second bonding location (lead 100a), and then is configured to form a second bond of the wire loop at lead 100a. This is made clear in FIG. 6 as die pad 102a is labelled “1” and lead 100a is labelled “2”. In keeping with teaching the bonding locations in the order in which they are to be wirebonded, the teaching process then continues to die pad 102b (labelled with a “3”) and then to lead 100b (labelled with a “4”) (a wire loop is configured to be extended between die pad 102b and lead 100b). Then the teaching process continues to die pad 102c (labelled with a “5”) and then to lead 100c (labelled with a “6”). Then the teaching process continues to die pad 102d (labelled with a “7”) and then to lead 100d (labelled with a “8”), and so on, until the teaching process teaches die pad 102l (labelled with a “23” and then lead 100l (labelled with a “24”). By teaching the bonding locations in the order in which they are to be wirebonded, a significant portion of the exemplary previously described potential error sources may be substantially limited or avoided.

FIG. 6 illustrates an exemplary motion path of the xy table (i.e., as indicated from labels “1” through “24”) in a given application. As provided above, the actual motion path during the teach process is the path in which the bonding locations are configured to be wire bonded. Thus, in contrast to the simplistic example shown in FIG. 6 (where the bonding locations are arranged in a “square” pattern, and are taught in a counterclockwise direction), the actual motion of the xy table during the teaching operation may be any of a number paths (e.g., back and forth motions, changing directions, fanning in and out motions, etc.). In another example, a stand-off stitch bond type wire loop (i.e., a wire loop including a conductive bump formed separate from the rest of the wire loop, where a portion of the rest of the wire loop is bonded to top of the bump) would tend to have a more complicated motion path than the motion path shown in connection with the teach process of FIG. 6.

As shown in FIG. 6, die pads 102a, 102b, and 102c are positioned in a row (i.e., along a distinct axis), and leads 100a, 100b, and 100c, are also positioned in a row (i.e., along a distinct axis). Likewise, die pads 102d, 102e, and 102f are also configured in a row (i.e. along a distinct axis), where this axis is distinct from (and in fact substantially perpendicular to) the axis along which die pads 102a, 102b, and 102c extend. Thus, according to certain aspects of the present invention, bonding locations may be taught in the order in which they are configured to be wire bonded, where the bonding locations include bonding locations that extend along at least two distinct axes on one or more of the components of the semiconductor device.

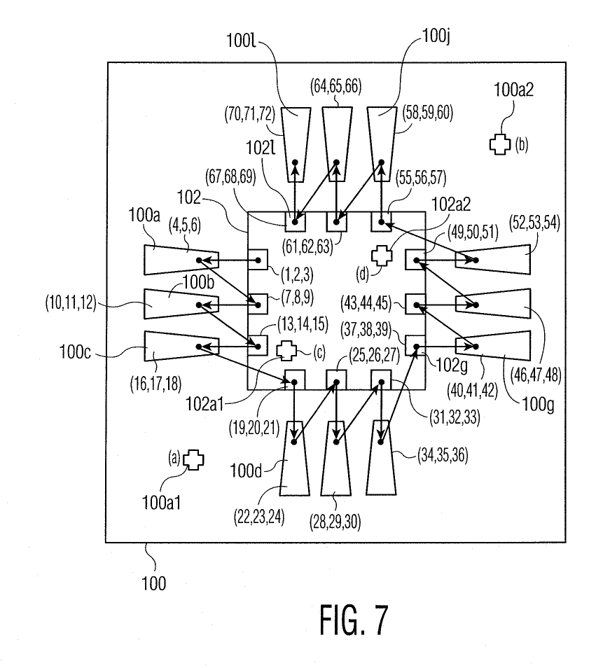

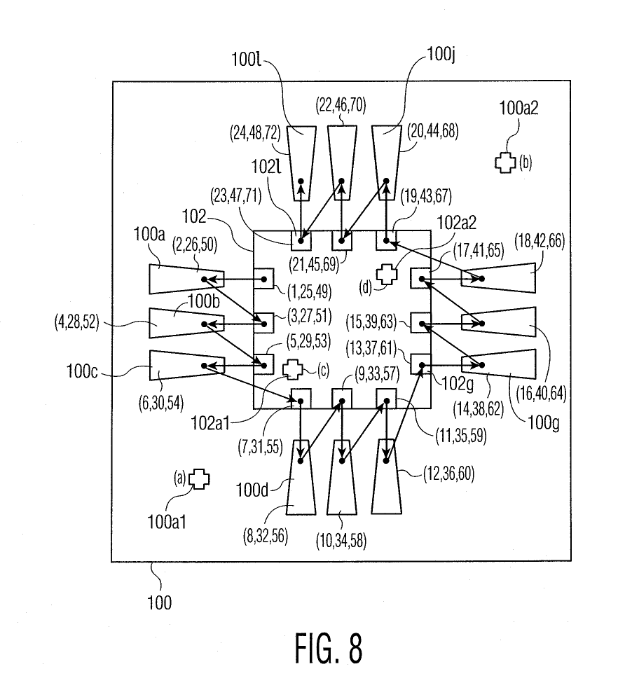

According to certain exemplary embodiments of the present invention, it may be desirable to scan each of the bonding locations multiple times during the teaching process (if desired, the eyepoints may also be scanned multiple times during the teach process), and as such the position data associated with the scans may be collectively used to determine more accurate position data for each of the bonding locations (and if desired, to provide more accurate position data for the eyepoints). For example, using the position data for each of the multiple scans of a given bonding location, more accurate position data for that bonding location may be arithmetically derived (e.g., by mathematically manipulating the position data of each scan by averaging or the like). There are various techniques through which multiple scans of each bonding location may be achieved. FIGS. 7-8 illustrate two such exemplary techniques. Following the teaching of the eyepoints in each of FIGS. 7-8 (in the exemplary order “a” through “d”), the bonding locations are taught as described below.

Referring specifically to FIG. 7, the illustrated semiconductor device includes semiconductor die 102 supported by leadframe 100. The eyepoints, leads and die pads are the same as those shown in FIGS. 2 and 6. FIG. 7 is similar to FIG. 6 in that it illustrates a teaching process for bonding locations (i.e., the die pads and leads) where the bonding locations are taught in the order in which they are configured to be wire bonded; however, FIG. 7 is different from FIG. 6 in that multiple scans/images are obtained of each bonding location during the teach process. In the example shown in FIG. 7, the wire bonding machine (e.g., the pattern recognition system of the wire bonding system) obtains multiple images of each of the bonding locations prior to moving to the next bonding location in the order in which the bonding locations are configured to be wire bonded. That is, at the first bonding location (i.e., die pad 102a) three images are taken as indicated by the label (“1, 2, 3”). Then the teaching process proceeds to the next bonding location (i.e., lead 100a) where three images are taken as indicated by the label (“4, 5, 6”). This teaching process continues in the order in which the bonding locations are configured to be wire bonded (as in FIG. 6), but three images are taken at each bonding location. Thus, in a single pass in the order in which the bonding locations are configured to be wire bonded, three images are taken of each bonding location. As will be described in greater detail below, these multiple images may be used collectively to arrive at a single more accurate representation of the position of each bonding location.

Referring specifically to FIG. 8, the illustrated semiconductor device includes semiconductor die 102 supported by leadframe 100. The eyepoints, leads and die pads are the same as those shown in FIGS. 2, 6, and 7. FIG. 8 is similar to FIG. 6 in that it illustrates a teaching process for bonding locations (i.e., the die pads and leads) where the bonding locations are taught in the order in which they are configured to be wire bonded; however, like FIG. 7, FIG. 8 is different from FIG. 6 in that multiple scans are taken of each bonding location during the teach process. In the example shown in FIG. 8, the wire bonding machine (e.g., the pattern recognition system of the wire bonding system) obtains multiple images of each of the bonding locations through multiple passes, where each pass is conducted in the order in which the bonding locations are configured to be wire bonded. That is, at the first bonding location (i.e., die pad 102a) three images are taken as indicated by the label (“1, 25, 49”). The next bonding location (i.e., lead 100a) illustrates that three images are taken as indicated by the label (“2, 26, 50”). Stated differently, in the first pass, an image is taken at die pad 102a (as indicated by the label “1”), then an image is taken at lead 100a (as indicated by the label “2”), then an image is taken at die pad 102b (as indicated by the label “3”), and so on, until the first pass is complete when an image is taken at lead 100l (as indicated by the label “24”). After the first pass is complete (with 24 total images taken, one for each bonding location), a second pass is conducted beginning with die pad 102a (as indicated by the label “25”), then an image is taken at lead 100a (as indicated by the label “26”), and so on, until the second pass is complete when an image is taken at lead 100l (as indicated by the label “48”). After the second pass is complete (with 24 total images taken, one for each bonding location), a third pass is conducted beginning with die pad 102a (as indicated by the label “49”), then an image is taken at lead 100a (as indicated by the label “50”), and so on, until the third pass is complete when an image is taken at lead 100l (as indicated by the label “72”). In between each pass the eyepoints (e.g., eyepoints 100a1, 100a2, 102a1, 102a2, or a portion of the eyepoints) may be scanned again (and the eyepoints may be scanned multiple times in connection with each pass to achieve more accurate position data for the eyepoints). Thus, through the three passes taken in the order in which the bonding locations are configured to be wire bonded, three images are taken of each bonding location. As will be described in greater detail below, these multiple images may be used collectively to arrive at a single more accurate representation of the position of each bonding location.

By using the exemplary techniques disclosed herein, improved position data for the bonding locations may be derived, and stored in the memory of a wire bonding machine. When it is time to wire bond a batch of devices, this improved position data may be used to bond the batch of devices without re-teaching any of the bonding locations. However, it may be desirable to teach the bonding locations of more than a single sample device.

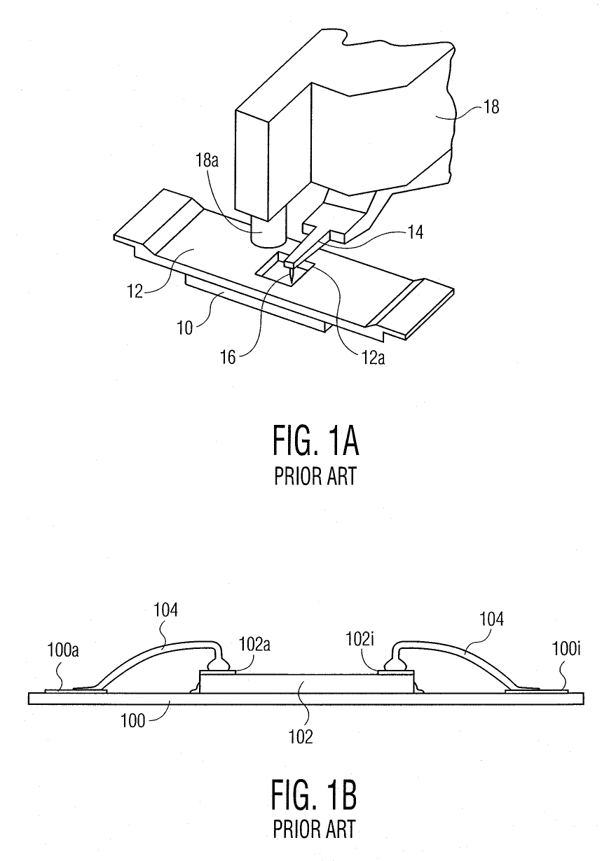

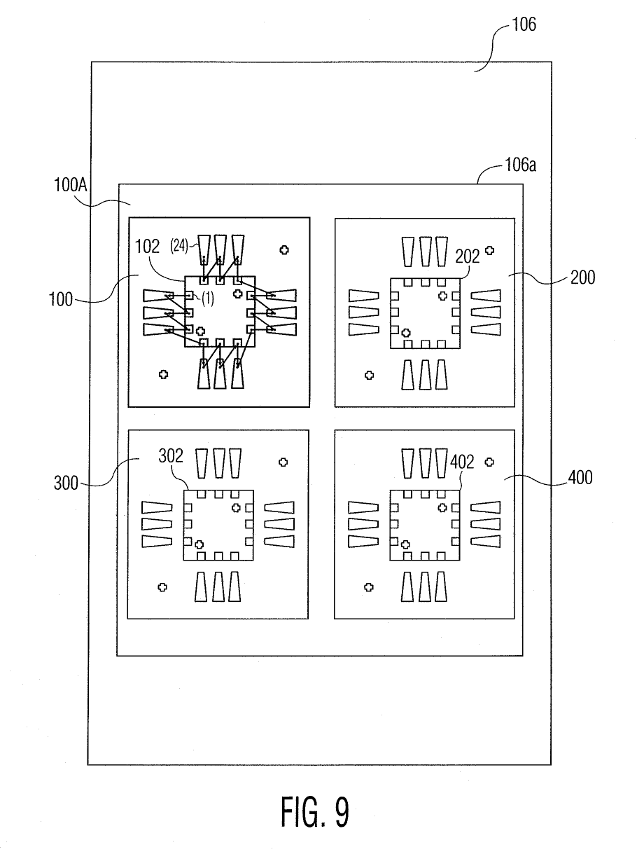

FIG. 9 is a top view of device clamp 106 (similar to device clamp 12 shown in FIG. 1A). Device clamp 106 defines aperture/window 106a through which devices to be wire bonded may be accessed using a bonding tool. As is known to those skilled in the art, a number of devices to be wire bonded may be on a leadframe strip, and the leadframe strip is indexed such that a portion of the devices to be wire bonded are positioned within the device clamp aperture. After this portion of the devices has been taught using a PRS, another portion of the devices on the leadframe strip may be positioned (using the wire bonding indexer system) within the device clamp aperture to be taught (or later wire bonded). Referring again to FIG. 9, leadframe strip 100A is positioned below device clamp 106 (only a portion of leadframe strip 100A is visible in FIG. 9). Through aperture 106a, a portion of the devices to be wire bonded on leadframe strip 100A are accessible for wire bonding. That is, semiconductor die 102 (supported by leadframe 100), semiconductor die 202 (supported by leadframe 200), semiconductor die 302 (supported by leadframe 300), and semiconductor die 402 (supported by leadframe 400) are accessible through aperture 106a. As illustrated in FIG. 9, the bonding locations on semiconductor die 102 and leadframe 100 have been taught in a manner similar to that illustrated in FIG. 6 (with the bonding locations being taught in the order in which they are configured to be wire bonded). This may be the sample device that is taught (or re-taught) according to an exemplary embodiment of the present invention. Thus, after more accurate position data of the bonding locations for semiconductor die 102 and leadframe 100 are taught as in FIG. 9, this more accurate position data may be applied to a batch of devices to be wire bonded (where the batch of devices may include semiconductor die 202 supported by leadframe 200, semiconductor die 302 supported by leadframe 300, and semiconductor die 402 supported by leadframe 400).

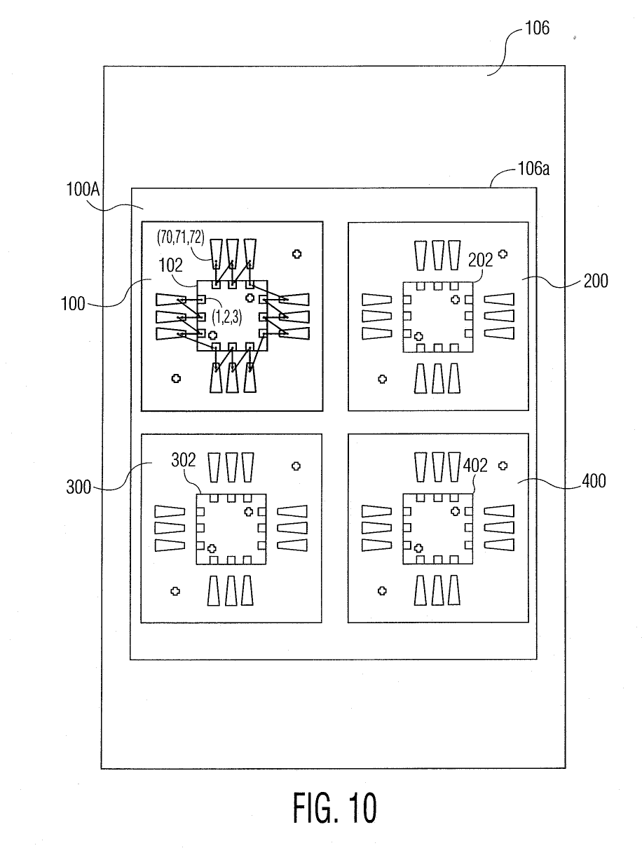

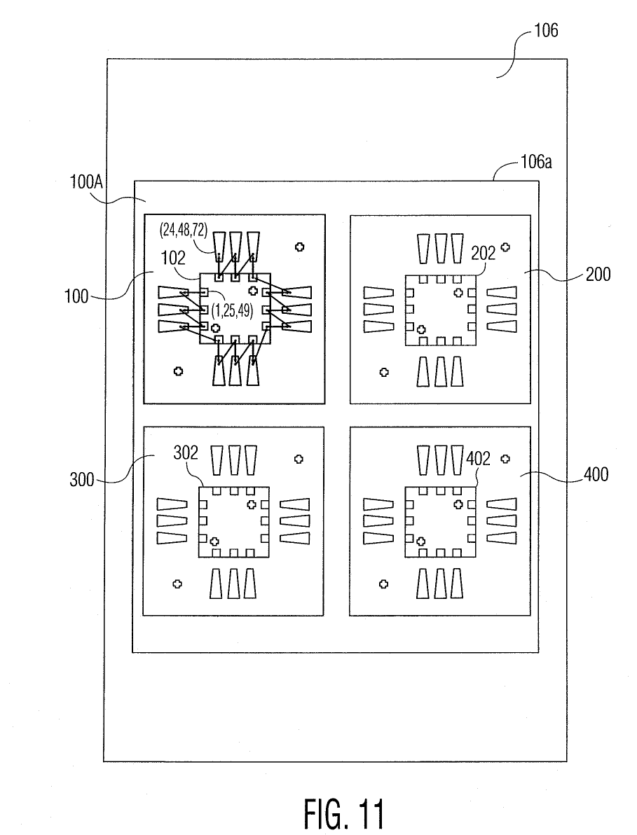

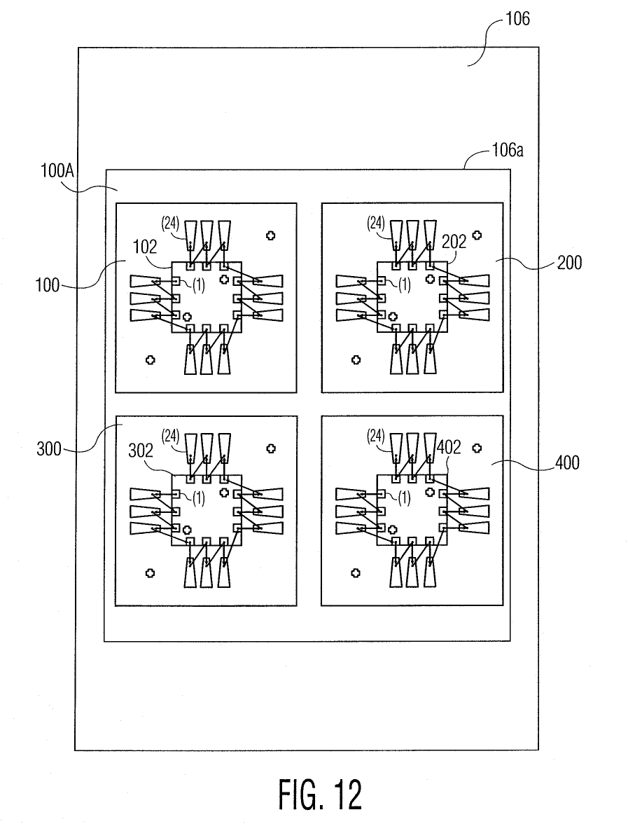

FIGS. 10-11 illustrate that the bonding locations of the sample device taught according to an exemplary embodiment of the present invention may be scanned multiple times, as described above in connection with FIGS. 7-8. That is, FIG. 10 illustrates the bonding locations of semiconductor die 102 and leadframe 100 being taught in the manner of the corresponding bonding locations of semiconductor die 102 and leadframe 100 shown in FIG. 7. Likewise, FIG. 11 illustrates the bonding locations of semiconductor die 102 and leadframe 100 being taught in the manner of the corresponding bonding locations of semiconductor die 102 and leadframe 100 shown in FIG. 8. Regardless of the exact methodology for performing multiple scans of each bonding location during the teach process of the sample device, the position data obtained during each of the multiple scans may collectively be utilized to obtain a more accurate representation of the actual bonding locations. Then, the position data obtained by utilizing the collective position data from each of the scans may be used when a batch of semiconductor devices is wire bonded (where the batch of devices may include semiconductor die 202 supported by leadframe 200, semiconductor die 302 supported by leadframe 300, and semiconductor die 402 supported by leadframe 400).

FIG. 12 illustrates that the bonding locations of more than one sample device may be taught according to the present invention in order to obtain more accurate position data for each of the bonding locations. That is, in FIG. 12, each of the four devices accessible though aperture 106a (i.e., semiconductor die 102 supported by leadframe 100, semiconductor die 202 supported by leadframe 200, semiconductor die 302 supported by leadframe 300, and semiconductor die 402 supported by leadframe 400) is taught according to inventive techniques. By teaching multiple devices, additional samples of the position data are obtained which may be utilized (e.g., through some type of statistical/mathematical analysis such as averaging) in order to achieve more accurate position data for each the bonding locations during an actual wire bonding process.

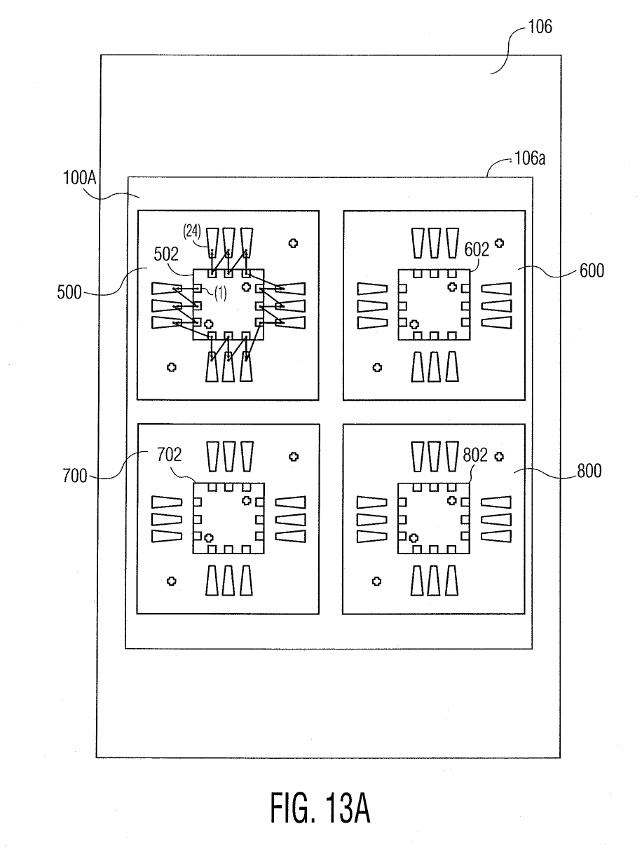

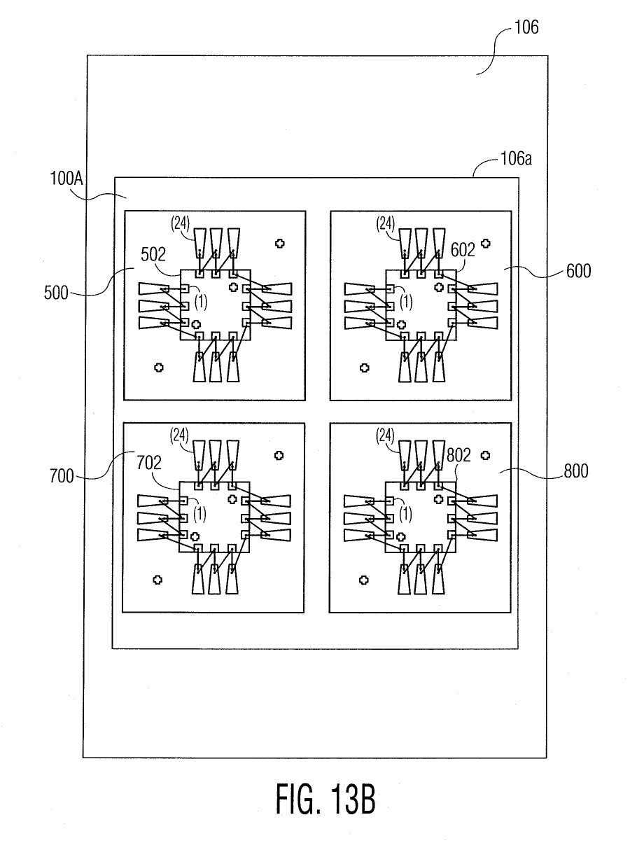

Like FIGS. 9-12, FIGS. 13A-13B illustrate device clamp 106 defining aperture 106a; however, FIGS. 13A-13B illustrate a different portion of leadframe strip 100A having been indexed into a position below aperture 106a of device clamp 106. That is, the four devices accessible though aperture 106a in FIGS. 13A-13B are semiconductor die 502 supported by leadframe 500, semiconductor die 602 supported by leadframe 600, semiconductor die 702 supported by leadframe 700, and semiconductor die 802 supported by leadframe 800. FIGS. 13A-13B illustrate further examples of additional teaching operations which may be performed in order to obtain more accurate position data for the bonding locations.

FIG. 13A illustrates one device (i.e., semiconductor die 502 supported by leadframe 500) that is taught in the manner shown in FIGS. 6 and 9. That is, FIG. 13A is intended to illustrate that after teaching one device (as in FIGS. 9, 10, and 11) or multiple devices (as in FIG. 12), where the one or multiple devices are accessible through the device clamp aperture, that an additional device (or additional devices in FIG. 13B) may be taught after indexing a new group of devices into the bonding position.

Thus, it is clear that there are various methods of improving the position data obtained by performing multiple teaching/scanning operations in accordance with the present invention. To summarize some of the methods that have been described: (1) a single sample device being taught may undergo multiple scans of each bonding location to obtain multiple samples of position data for each bonding location (e.g., as in FIGS. 7-8 and 10-11); multiple sample devices may be scanned one time each to obtain multiple examples of position data for each bonding location (e.g., as in FIGS. 12 and 13B); and multiple sample devices may undergo multiple scans of each bonding location to obtain multiple samples of position data for each bonding location (e.g., combining the teachings of FIGS. 7-8 and 10-11 with the teachings of FIGS. 12 and 13B). Of course, other variations are contemplated. No matter which technique is used, various samplings of position data for a given bonding location are obtained. An exemplary use of these various samplings is to arithmetically derive more accurate position data useful for when the actual wire bonding operation (e.g., for a batch of devices) is to be performed.

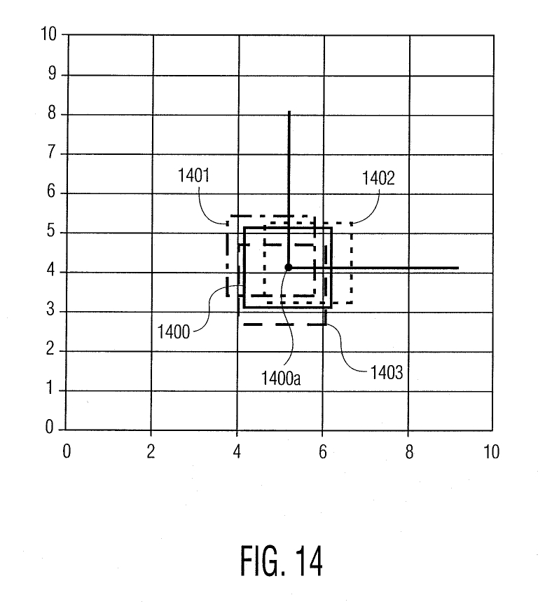

FIG. 14 is an illustration which is useful to explain an exemplary technique for using the various samplings to arithmetically derive more accurate position data for use when an actual wire bonding operation is to be performed. Consider again the example shown in FIG. 7 where each bonding location undergoes 3 scans in a single pass. Thus, three images are taken of each bonding location. If we consider the three images taken of die pad 102a: one image of die pad 102a may be image 1401 in FIG. 14; another image of die pad 102a may be image 1402 in FIG. 14; and yet another image of die pad 102a may be image 1403 in FIG. 14. The three images (i.e., 1401, 1402, and 1403) are plotted on a set of coordinate axes in FIG. 14 for mathematical illustration only. Thus, each of these images has a position on the coordinate axes (where the coordinate axes is illustrative of a position within the coordinate system of the semiconductor die on the wire bonding machine). The position data may be described in any of a number of manners (e.g., top edge of die pad, bottom edge of die pad, left edge of die pad, right edge of die pad, center of die pad, combinations thereof, amongst others). If we consider the position data to be represented by the center of each die pad, the position data (in terms of x,y coordinates) for image 1401 is (x=4.8, y=4.4); the position data for image 1402 is (x=5.7, y=4.2); and the position data for image 1403 is (x=5.1, y=3.7). The collective position data may then be mathematically manipulated in order to arithmetically derive more accurate position data for die pad 102a. For example, the collective position data may be averaged to derive more accurate position data for die pad 102a.

An exemplary expression for averaging the collective position data is:

$$\overline{x} = \frac{{\sum }_{i = 1}^{N} ( {x}_{i} )}{N}$$

$$\overline{y} = \frac{{\sum }_{i = 1}^{N} ( {y}_{i} )}{N}$$

where an average x position is determined (using the x position of the center point of each image), and where an average of the y position is determined (using the y position of the center point of an each image). Plugging the position data from the 3 data points into the exemplary expression above, the following relation is provided:

$$\overline{x} = \frac{5.1 + 5.7 + 4.8}{3} = 5.20$$

$$\overline{y} = \frac{3.7 + 4.2 + 4.4}{3} = 4.10$$

Thus, the position data (where in this example, the position data is calculated by calculating a centerpoint average of each image) is (x=5.2, y=4.1). This centerpoint is illustrated in FIG. 14 as point 1400a, and the average position of the entire die pad is illustrated as solid box 1400.

Thus, as described above in connection with FIG. 14, the various scans obtained through any of a number of techniques may be averaged or otherwise mathematically manipulated to arithmetically derive more accurate position data for each bonding location. This more accurate position data (for each bonding location) may then be saved to the memory of the wire bonding machine (e.g., in a bond program) to be used when wire bonding a batch of semiconductor devices. Similar techniques may be employed to provide more accurate position data for eyepoints when multiple scans of the eyepoints have been obtained.

While the example described above in connection with FIG. 14 was described in connection with the example shown in FIG. 7 (where there are 3 images taken of each bonding location in a single pass) it is clear that this is an example. Thus, the techniques described above in connection with FIG. 14 (or any other mathematical manipulation techniques) may be applied to multiple images taken using any of a number of techniques. Further, while 3 images are used in connection with the example in FIG. 14, any number of images may be taken and utilized in the arithmetic derivation of more accurate position data for each bonding location.

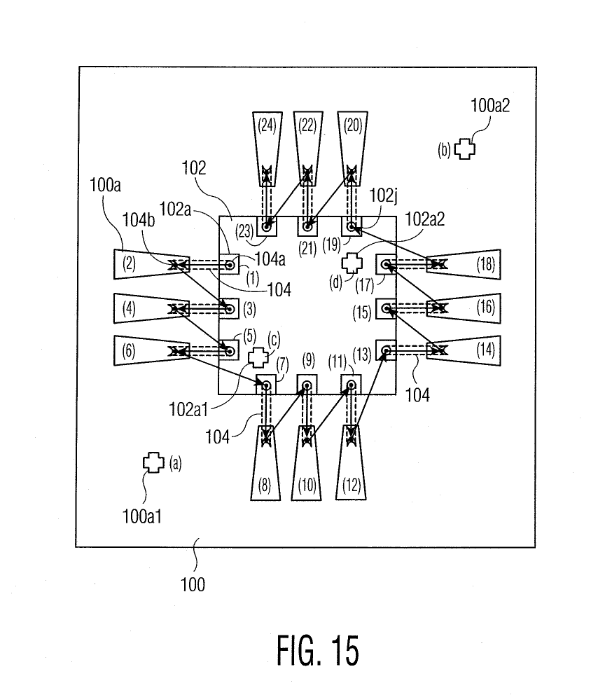

The benefits achieved using the teaching techniques of the present invention (e.g., substantially limiting the effect of the error sources described above) are also applicable to inspection techniques (e.g., PBI) of wire loops that have already been formed. For example, FIG. 15 illustrates semiconductor die 102 supported by leadframe 100 (as in FIGS. 2, 6, etc), where wire loops 104 provide electrical interconnection between respective die pads (e.g., die pads 102a, 102b, etc.) and leads (e.g., leads 100a, 100b, etc.). Each wire loop includes a respective first bond portion (e.g., ball bond 104a) formed on a die pad of semiconductor die 102, and a second bond portion (e.g., stitch bond portion 104b) formed on a lead of leadframe 100. Often it is desired to examine the first bond portion (a ball bond portion) of a wire loop. For example, it may be desirable to determine the diameter of the ball bond portion, the position of the ball bond portion with respect to the die pad (e.g., where the die pad position may be determined by scanning the eyepoints, where the die pad position may be known from the teaching techniques disclosed herein, etc.), amongst other information about the first bond portion and each eyepoint location.

In FIG. 15, the inspection of wire loops 104 follows the path in which the wire loops were wire bonded in order to substantially limit the potential for certain of the previously described error sources. Thus, the imaging equipment of the PRS moves in the order from 1-24, as illustrated in FIG. 15 (e.g., the operation illustrated in FIG. 15 may be conducted after scanning the eyepoints 100a1, 100a2, 102a1, 102a2, once each or multiple times each). In this regard, the PRS first moves to die pad 102a of semiconductor die 102 (as indicated by the label “1”), and then the PRS moves to lead 100a of leadframe 100 (as indicated by the label “2”), and then to die pad 102b (as indicated by the label “3”), and so on, until the PRS reaches lead 100l (as indicated by the label “24”). While in certain applications it may be desirable to obtain image/position data for each bonding location (including the first bonding locations and the second bonding locations), in certain embodiments it may be desired to only inspect a portion of the wire loop on certain of the bonding locations. For example, it may only be desired to inspect the first bond portion of the wire loops (e.g., in FIG. 15 the first bond portion of the wire loops is a ball bond portion formed on each die pads of semiconductor die 102). Nonetheless, it may be beneficial to move the imaging equipment of the PRS in the manner illustrated in FIG. 15 (which includes the motions to the second bonding locations).

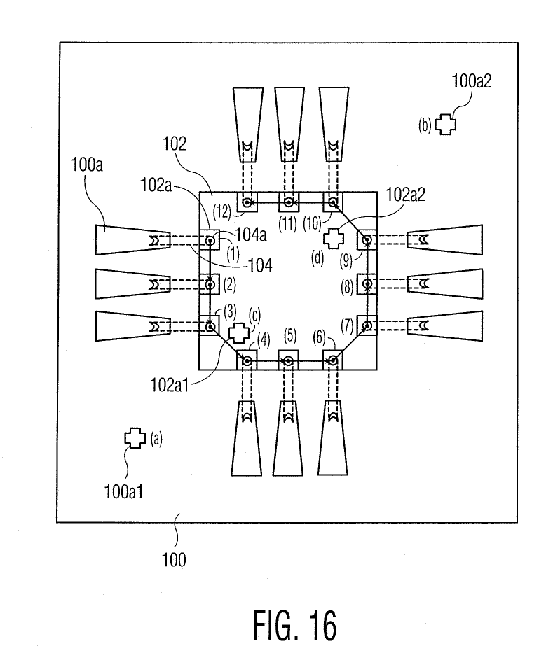

Alternatively, in another example shown in FIG. 16, it may be desired to move the relevant portions of the PRS system only to those bonding locations to be inspected. In FIG. 16, after any desired alignment/re-alignment conducted by scanning of the eyepoints (e.g., eyepoints 100a1, 100a2, 102a1, 102a2), the motion path begins at die pad 102a (as indicated by the label “1”), then continues to die pad 102b (as indicated by the label “2”), then continues to die pad 102c (as indicated by the label “3”), and so on, until the final image is taken at die pad 102l (as indicated by the label “12”). The path illustrated in FIG. 16 does not follow the path in which the wire loops were formed (as in FIG. 16), and as such does not provide certain benefits related to correction of potential error sources. Nonetheless, many of the aforementioned benefits are still provided if the path utilizes bonding location position data previously obtained, for example, through the inventive teaching techniques disclosed herein.

The inspection techniques disclosed herein may also be repeated in a manner previously described with respect to the teaching of the bonding locations. For example, multiple images of the predetermined portions of the wire loops to be inspected may be taken in a single pass (as described in connection with teaching bonding locations in FIG. 7); multiple images of the predetermined portions of the wire loops to be inspected may be taken in multiple passes (as described in connection with teaching bonding locations in FIG. 8); multiple images of the predetermined portions of the wire loops to be inspected may be taken by obtaining multiple images in each pass, where multiple passes are taken (i.e., a combination of the techniques described in connection with teaching bonding locations in FIGS. 7-8), amongst others.

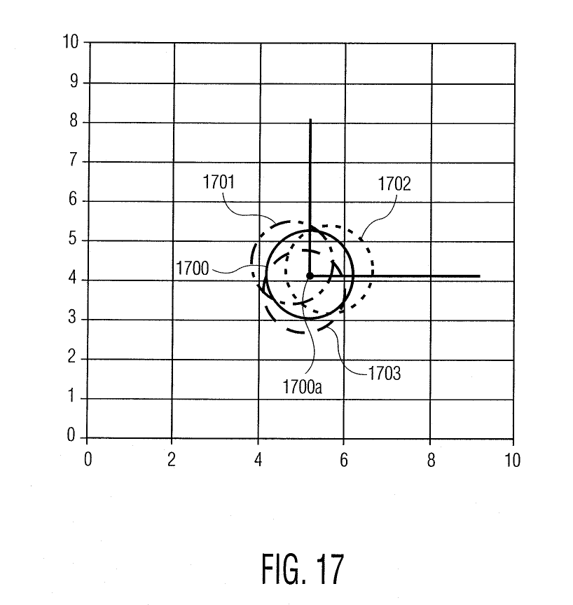

FIG. 17 is an illustration which is useful to explain an exemplary technique for using the various samplings to arithmetically derive more accurate position data for use in inspecting a portion of a wire loop. As previously described in FIG. 14, consider an example where each first ball bond portion (a substantially circular image) of the scanned wire loops undergoes 3 scans in a single pass. Thus, three images are taken of each first ball bond portion. If we consider the three images taken of the ball bond portion of a given wire loop (e.g., ball bond portion 104a of wire loop 104 shown in FIG. 15), the images are labeled in FIG. 17 as 1701, 1702, and 1703. The three images (i.e., 1701, 1702, and 1703) are plotted on a set of coordinate axes in FIG. 17 for mathematical illustration only. Thus, each of these images has a position on the coordinate axes. The position data may be described in any of a number of manners (e.g., center of ball bond, radius of ball bond from center, diameter of ball bond, combinations thereof, amongst others). If we consider the position data to be represented by the center of each ball bond, then each center point may be obtained in terms of x and y coordinates as described above in connection with FIG. 14. These x,y positions may then be mathematically manipulated (e.g., averaged) in order to arithmetically derive more accurate position data (e.g., a centerpoint) for each ball bond. The centerpoint of the ball bond is illustrated in FIG. 17 as point 1700a, and the average of the entire ball bond is illustrated as solid circle 1700. By obtaining more accurate position data of the ball bond during PBI (through the mathematical manipulation of the multiple scans of the ball bond portion), more accurate PBI results may be achieved.

By providing improved inspection data according to the various exemplary embodiments of the present invention described herein, a number of benefits may be achieved. For example, as is known to those skilled in the art, there is an offset between the bonding tool (e.g., bonding tool 16 in FIG. 1) and the portion of the optics assembly carried by the bond head adjacent the bonding tool (e.g., camera portion 18a in FIG. 1). It is important that the offset (sometimes referred to as a “crosshair offset”) is known to a high degree of accuracy. For example, after the teaching process is conducted (e.g., teaching of the bonding locations and eyepoints using camera portion 18a), the bond head of the wire bonding machine is moved to use the bonding tool to perform the wire bonding operation. If the offset is not accurately known, the bonding tool will not be in a desired position for the wire bonding operation, resulting in wire bonds formed in a potentially undesirable location on a die pad or the like. This is further complicated because potential errors associated with the offset are different when performing (1) an imaging operation (using the PRS), versus (2) a wire bonding operation (using the bonding tool). Further, the offset may change over time (during either of a teach process or a wire bonding process) because of temperature influences and the like. By deriving more accurate inspection data in accordance with the present invention, certain inaccuracies in the offset may be accounted for, thereby providing for a more accurate wire bonding process.

Although the inspection techniques described above primarily relate to inspection of the first bond portion of wire loops, the present invention is not limited thereto. The inventive techniques may be applied to various portions of wire loops (e.g., second bond portions).

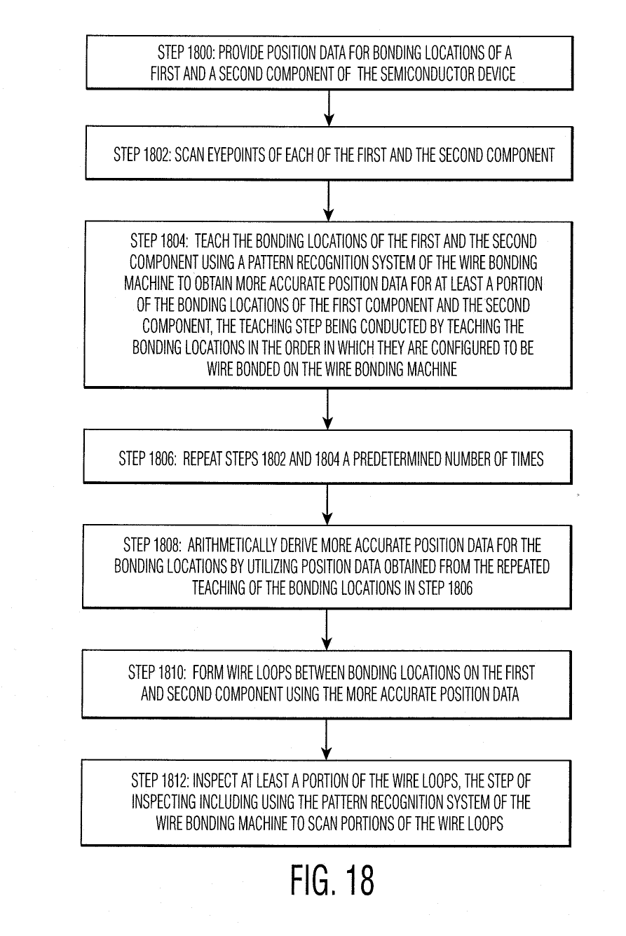

FIG. 18 is a flow diagram illustrating various exemplary embodiments of the present invention. As is understood by those skilled in the art, certain steps included in the flow diagram may be omitted; certain additional steps may be added; and the order of the steps may be altered from the order illustrated.

More specifically, the flow diagram in FIG. 18 includes (1) steps of teaching bonding locations of a semiconductor device, and (2) steps of forming and inspecting wire loops. At step 1800, the wire bonding machine is provided with position data for (1) bonding locations of a first component of the semiconductor device, and (2) bonding locations of a second component of the semiconductor device. For example, referring to the semiconductor device illustrated in FIG. 6 (where the first and second component are semiconductor die 102 and leadframe 100), position data may be provided for the die pads of semiconductor die 102 and for the leads of leadframe 100. This data may be provided, for example, through a teach process, or may be provided by offline data (e.g., CAD data or the like). At step 1802, the eyepoints of each of the first and the second component are scanned using the PRS system of the wire bonding machine. Again, referring to the example shown in FIG. 6, leadframe eyepoints 100a1 and 100a2, as well as eyepoints 102a1 and 102a2, may be taught in a predetermined order by the PRS.

At step 1804, the bonding locations of the first component of the semiconductor device and the second component of the semiconductor device are taught using a PRS of the wire bonding machine to obtain more accurate position data for at least a portion of the bonding locations of the first component and the second component. The teaching step is conducted by teaching the bonding locations in the order in which they are configured to be wire bonded on the wire bonding machine. For example, FIG. 6 illustrates an order of teaching the bonding locations beginning at die pad 102a (labelled “1”) and ending at lead 100l (labelled “24”). At step 1806, the teaching step of step 1804 (and the eyepoint scanning step of 1802) is repeated a predetermined number of times. For example, referring to FIGS. 7-8, two examples of repeating the teaching process shown in FIG. 6 are illustrated. At step 1808, more accurate position data is arithmetically derived for the bonding locations using the position data obtained from the repeated teaching of the bonding locations. For example, FIG. 14 illustrates a method for averaging the position data obtained from 3 scans of a given bonding location.

At step 1810, wire loops are formed between the bonding locations on the first and second component using the more accurate position data. For example, FIG. 15 illustrates wire loops 104 providing electrical interconnection between respective ones of the die pads of semiconductor die 102 and leads of leadframe 100. At step 1812, at least a portion of the wire loops are inspected using the PRS of the wire bonding machine. For example, FIGS. 15-16 illustrate exemplary techniques for scanning portions (e.g., first ball bond portions) of wire loops 104.

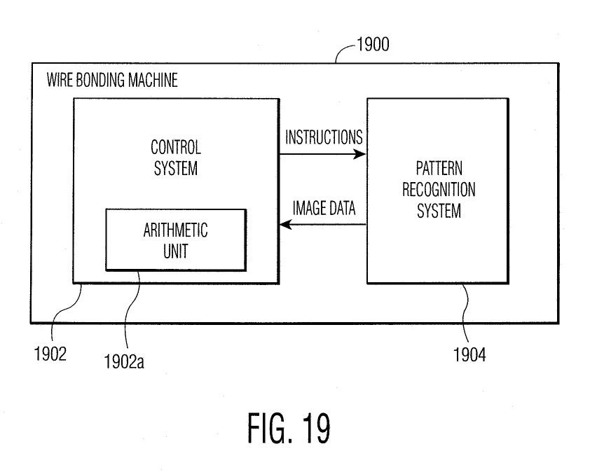

FIG. 19 is a block diagram of portion 1900 of the intelligence of a wire bonding machine which may be used in connection with certain exemplary techniques of the present invention. Portion 1900 of the wire bonding machine includes control system 1902 and pattern recognition system 1904. Pattern recognition system 1904 is configured for teaching (a) bonding locations of a first component of a semiconductor device (such as die pads of semiconductor die 102), and (b) bonding locations of a second component of the semiconductor device (such as leads of leadframe 100). Control system 1902 includes arithmetic unit 1902a. Control system 1902 is configured to control operation of pattern recognition system 1904 such that pattern recognition system 1904 teaches the bonding locations of the first component and the second component in the order in which the bonding locations are configured to be wire bonded. In this regard, and as illustrated in FIG. 19, certain information passes between control system 1902 and pattern recognition system 1904. For example, control system 1902 sends instructions to pattern recognition system 1904 regarding operation of pattern recognition system 1904. Additionally, pattern recognition system 1904 sends image data to control system 1902. If multiple images are taken of the bonding locations in a teaching process (or if multiple images are taken of predetermined portions of wire loops in an inspection process), the image data may be used by arithmetic unit 1902a to arithmetically derive more accurate position data (or inspection data in an inspection process). Of course, these components are exemplary in nature and may be provided in a number of forms, as is known to those skilled in the art of wire bonding machines. For example, portions of pattern recognition system 1904 may be considered to be part of control system 1902.

Although the present invention has been described primarily with respect to teaching operations conducted on one or more sample devices, where the teaching operations are followed by a wire bonding operation being performed on a batch of semiconductor devices (where the wire bonding operations uses the more accurate position data derived from the teaching of the sample device(s)), it is not limited thereto. According to the present invention, it may be desirable to perform certain of the inventive teaching techniques at different intervals during a wire bonding process to account for system changes (e.g., temperature shifts, mechanical shifts, etc.). Thus, it may be desirable to perform a re-teaching operation (using any of the inventive techniques disclosed or claimed herein) at a predetermined interval. For example, such a predetermined interval may be a time based interval (e.g., every 15 minutes during wire bonding, every 6 hours during wire bonding, etc.), a wire loop count based interval (e.g., every one thousand wire loops formed during wire bonding, etc), a device based interval (every 100 devices that have been wire bonded, etc.), amongst others. By performing a re-teaching operation at certain intervals, improved position data may be derived that is more applicable to the actual current status of the wire bonding machine and the devices to be wire bonded.

Certain exemplary embodiments of the present invention have been described herein in connection with teaching bonding locations (and/or eyepoints) in the order in which they are configured to be wire bonded. In connection with such embodiments of the present invention, the xy table path direction and distance may be the same during teaching as it is configured to be during wire bonding. However, in certain embodiments of the present invention, certain other characteristics of the xy table motion during the teaching process may follow the xy table motion configured for the wire bonding process. For example, the velocity, acceleration, and motion time for certain of the motions during the teaching process may follow the xy table motion configured for the wire bonding process. This may provide an improved level of accuracy in certain applications; however, it may not be practical in certain operations. For example, during wire looping motions from a first bonding location to a second bonding location (e.g., from die pad 102a to lead 100a) the velocity of the xy table tends to vary during different portions of the wire looping cycle. Further, this may result in a relatively long time for the wire bonding/looping operation. This level of complexity (and loss of time) may not be desirable during teaching operations. Nonetheless, such an approach may be taken in other motions (e.g., the motion after completing a wire loop to the first bond location of the next wire loop, the motion from an eyepoint to a first bond location, etc.) if desired.

Certain exemplary embodiments of the present invention have been described in connection with a teaching operation whereby the eyepoints are taught/scanned, and then the bonding locations are taught/scanned in the order in which they are configured to be wire bonded; however, as will be appreciated by those skilled in the art, during the teaching operation, after the eyepoints are scanned, the motion from the eyepoint to the first bonding location will tend to be different from the corresponding motion during the wire bonding operation. This is because of the previously described “offset” between the camera portion and the bonding tool. During a wire bonding operation, the motion from the final eyepoint scan (where the camera portion is above the eyepoint) to the first bonding location (where the bonding tool is above the first bonding location) is a motion where the desired positional control point of the motion changes from the camera portion to the bonding tool. However, this is not the case during the teaching sequence because the camera portion is the desired positional control point of the motion at the eyepoint and at the first bonding location during the teaching operation. Therefore, in certain applications it may be desirable to correct for this offset in connection with the motion from the final eyepoint scan to the first bonding location during the teaching operation.

Although various illustrations provided herein illustrate each bonding location being taught during a teach process, and each bonded wire portion being inspected during an inspection process, the present invention is not limited thereto. During the teach process, it is clear that only a portion of the bonding locations may be actually taught. Likewise, during an inspection operation, it is clear that only a portion of the bonded portions (on a portion of the bonded wires) may be actually inspected.

The present invention may be implemented in a number of alternative mediums. For example, the techniques can be installed on an existing computer system/server as software (a computer system used in connection with, or integrated with, a wire bonding machine). Further, the techniques may operate from a computer readable carrier (e.g., solid state memory, optical disc, magnetic disc, radio frequency carrier medium, audio frequency carrier medium, etc.) that includes computer instructions (e.g., computer program instructions) related to the inventive techniques.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A method of teaching bonding locations of a semiconductor device on a wire bonding machine, the method comprising the steps of:
(1) providing the wire bonding machine with position data for (1) bonding locations of a first component of the semiconductor device, and (2) bonding locations of a second component of the semiconductor device; and
(2) teaching the bonding locations of the first component of the semiconductor device and the second component of the semiconductor device using a pattern recognition system of the wire bonding machine to obtain more accurate position data for at least a portion of the bonding locations of the first component and the second component, the teaching step being conducted by teaching the bonding locations in the order in which they are configured to be wire bonded on the wire bonding machine.

2. The method of claim 1 wherein the teaching step includes teaching the bonding locations that extend along at least two distinct axes on at least one of the first component and the second component.

3. The method of claim 1 wherein the first component is a semiconductor die and the second component is a substrate on which the semiconductor die is mounted, the teaching step including teaching the bonding locations on each of the semiconductor die and the substrate in the order in which they are configured to be wire bonded on the wire bonding machine.

4. The method of claim 1 further comprising the step of scanning eyepoints of each of the first component and the second component after step (1) but before step (2).

5. The method of claim 1 wherein the teaching step includes repeating the teaching of the bonding locations a predetermined number of times.

6. The method of claim 5 further comprising the step of scanning eyepoints of each of the first component and the second component prior to each of the repeated teaching steps.

7. The method of claim 5 wherein at least a portion of the repeating of the teaching of the bonding locations is conducted successively in a single pass conducted in the order in which the bonding locations are configured to be wire bonded on the wire bonding machine, whereby the pattern recognition system obtains multiple images of each of the bonding locations prior to moving to the next bonding location in the order in which the bonding locations are wire bonded.

8. The method of claim 5 wherein at least a portion of the repeating of the teaching of the bonding locations is conducted successively in multiple passes conducted in the order in which the bonding locations are configured to be wire bonded on the wire bonding machine, whereby the pattern recognition system obtains at least one image of each of the bonding locations during each of the multiple passes.

9. The method of claim 5 further comprising the step of arithmetically deriving more accurate position data for the bonding locations by utilizing position data obtained from the repeated teaching of the bonding locations.

10. The method of claim 9 wherein the step of arithmetically deriving more accurate position data includes averaging the position data for each of the bonding locations from each of the teaching steps of the repeated teaching.

11. The method of claim 1 further comprising the step of forming wire loops between bonding locations on the first component and bonding locations on the second component using the more accurate position data.

12. The method of claim 11 further comprising the step of inspecting at least a portion of the wire loops, the step of inspecting including using the pattern recognition system of the wire bonding machine to scan portions of the wire loops.

13. The method of claim 12 wherein the step of inspecting includes using the pattern recognition system of the wire bonding machine to scan a first bond portion of the portion of the wire loops, the step of inspecting being conducted by scanning the first bond portion of the wire loops successively and in order without inspecting any other portion of the wire loops.

14. The method of claim 12 wherein the step of inspecting includes using the pattern recognition system of the wire bonding machine to scan portions of the wire loops at respective bonding locations of the wire loops in the order in which they were wire bonded on the wire bonding machine.

15. The method of claim 14 wherein the step of inspecting includes scanning a first ball bond portion of at least a portion of the wire loops and comparing a scanned position of the scanned ball bond portion with a desired position of the first ball bond portion on the respective bonding location.

16. The method of claim 14 wherein the step of inspecting is repeated a predetermined number of times, wherein the repeating of the inspecting step is (a) conducted successively in a single pass conducted in the order in which the bonding locations were wire bonded on the wire bonding machine, whereby the pattern recognition system obtains multiple images of each of the bonding locations prior to moving to the next bonding location in the order in which the bonding locations were wire bonded, or (b) conducted successively in multiple passes conducted in the order in which the bonding locations were wire bonded on the wire bonding machine, whereby the pattern recognition system obtains at least one image of each of the bonding locations during each of the multiple passes.

17. A computer readable carrier including computer program instructions which cause a computer to implement a method of teaching bonding locations of a semiconductor device on a wire bonding machine, the method comprising the steps of:
(1) providing the wire bonding machine with position data for (1) bonding locations of a first component of the semiconductor device, and (2) bonding locations of a second component of the semiconductor device; and
(2) teaching the bonding locations of the first component of the semiconductor device and the second component of the semiconductor device using a pattern recognition system of the wire bonding machine to obtain more accurate position data for at least a portion of the bonding locations of the first component and the second component, the teaching step being conducted by teaching the bonding locations in the order in which they are configured to be wire bonded on the wire bonding machine.

18. A wire bonding machine comprising:
a pattern recognition system for teaching (a) bonding locations of a first component of a semiconductor device, and (b) bonding locations of a second component of the semiconductor device; and
a control system configured to control operation of the pattern recognition system such that the pattern recognition system teaches the bonding locations of the first component and the second component in the order in which the bonding locations are configured to be wire bonded.

19. A method of teaching bonding locations of a semiconductor device on a wire bonding machine, the method comprising the steps of:
(1) teaching a plurality of bonding locations of a first component of the semiconductor device and a second component of the semiconductor device using a pattern recognition system of the wire bonding machine, the teaching step being conducted by teaching the bonding locations in the order in which they are configured to be wire bonded on the wire bonding machine, the teaching step including repeating the teaching of the bonding locations a predetermined number of times to obtain position data for each of the bonding locations for each of the repeated steps of teaching; and
(2) arithmetically deriving more accurate position data for the bonding locations by utilizing position data obtained from the repeated teaching of the bonding locations.

20. The method of claim 19 wherein at least a portion of the repeating of the teaching of the bonding locations is conducted successively in a single pass conducted in the order in which the bonding locations are configured to be wire bonded on the wire bonding machine, whereby the pattern recognition system obtains multiple images of each of the bonding locations prior to moving to the next bonding location in the order in which the bonding locations are wire bonded.

21. The method of claim 19 wherein at least a portion of the repeating of the teaching of the bonding locations is conducted successively in multiple passes conducted in the order in which the bonding locations are configured to be wire bonded on the wire bonding machine, whereby the pattern recognition system obtains at least one image of each of the bonding locations during each of the multiple passes.

22. The method of claim 19 wherein the step of arithmetically deriving more accurate position data includes averaging the position data for each of the bonding locations obtained from each of the teaching steps of the repeated teaching.

23. The method of claim 19 wherein the teaching step includes teaching the bonding locations that extend along at least two distinct axes on at least one of the first component and the second component.

24. The method of claim 19 further comprising the step of providing the wire bonding machine with position data for the bonding locations of the first component and the second component of the semiconductor device prior to step (1).

25. The method of claim 19 wherein the first component is a semiconductor die and the second component is a substrate on which the semiconductor die is mounted, the teaching step including teaching the bonding locations on each of the semiconductor die and the substrate in the order in which they are configured to be wire bonded on the wire bonding machine.

26. The method of claim 19 further comprising the step of scanning eyepoints of each of the first component and the second component prior to each of the repeated teaching steps.

27. The method of claim 19 further comprising the step of forming wire loops between bonding locations on the first component and bonding locations on the second component using the more accurate position data.

28. The method of claim 19 further comprising the step of inspecting at least a portion of the wire loops, the step of inspecting including using the pattern recognition system of the wire bonding machine to scan portions of the wire loops.

29. The method of claim 28 wherein the step of inspecting includes using the pattern recognition system of the wire bonding machine to scan a first bond portion of the portion of the wire loops, the step of inspecting being conducted by scanning the first bond portion of the wire loops successively proceeding from one of the first bond portions to another of the first bond portions.

30. The method of claim 28 wherein the step of inspecting includes using the pattern recognition system of the wire bonding machine to scan portions of the wire loops at respective bonding locations of the wire loops in the order in which they were wire bonded on the wire bonding machine.

31. The method of claim 30 wherein the step of inspecting includes scanning a first ball bond portion of at least a portion of the wire loops and comparing a scanned ball bond position of the scanned ball bond portion with a desired position of the first ball bond portion on the respective bonding location.

32. The method of claim 30 wherein the step of inspecting is repeated a predetermined number of times, wherein the repeating of the inspecting step is (a) conducted successively in a single pass conducted in the order in which the bonding locations were wire bonded on the wire bonding machine, whereby the pattern recognition system obtains multiple images of each of the bonding locations prior to moving to the next bonding location in the order in which the bonding locations were wire bonded, or (b) conducted successively in multiple passes conducted in the order in which the bonding locations were wire bonded on the wire bonding machine, whereby the pattern recognition system obtains at least one image of each of the bonding locations during each of the multiple passes.

33. A computer readable carrier including computer program instructions which cause a computer to implement a method of teaching bonding locations of a semiconductor device on a wire bonding machine, the method comprising the steps of:
(1) providing the wire bonding machine with position data for (1) bonding locations of a first component of the semiconductor device, and (2) bonding locations of a second component of the semiconductor device; and
(2) teaching the bonding locations of the first component of the semiconductor device and the second component of the semiconductor device using a pattern recognition system of the wire bonding machine to obtain more accurate position data for at least a portion of the bonding locations of the first component and the second component, the teaching step being conducted by teaching the bonding locations in the order in which they are configured to be wire bonded on the wire bonding machine.

34. A wire bonding machine comprising:
a pattern recognition system for teaching (a) bonding locations of a first component of a semiconductor device, and (b) bonding locations of a second component of the semiconductor device; and
a control system configured to control operation of the pattern recognition system such that (1) the pattern recognition system teaches the bonding locations of the first component and the second component in the order in which the bonding locations are configured to be wire bonded, and (2) the pattern recognition system repeats the teaching of the bonding locations a predetermined number of times to obtain position data for each of the bonding locations for each of the repeated steps of teaching, whereby the control system is configured to arithmetically derives more accurate position data for the bonding locations by utilizing the position data obtained from the repeated teaching of the bonding locations.

35. A method of inspecting wire loops of a semiconductor device on a wire bonding machine, the method comprising the steps of:
(1) providing a semiconductor device including a plurality of wire loops, each of the wire loops providing electrical interconnection between a first bonding location of the semiconductor device and a second bonding location of the semiconductor device; and
(2) inspecting predetermined portions of the wire loops using a pattern recognition system of the wire bonding machine, the inspecting step being conducted by moving a portion of the pattern recognition system to scan the predetermined portions of the wire loops at the respective bonding locations in the order in which they were wire bonded on the wire bonding machine.

36. The method of claim 35 wherein the semiconductor device includes a semiconductor die and a substrate on which the semiconductor die is mounted, the inspecting step including inspecting predetermined portions of the wire loops including a first bond portion of the wire loops, and wherein the first bond portion of the wire loops is formed on the semiconductor die or on the substrate.

37. The method of claim 35 wherein the semiconductor device includes a first component including the first bonding location and a second component including the second bonding location, the method further comprising the step of scanning eyepoints of each of the first component and the second component after step (1) but before step (2).

38. The method of claim 35 wherein the inspecting step includes repeating the inspecting of the predetermined portions of the wire loops a predetermined number of times, whereby inspection data is obtained during each of the repeated inspection steps.

39. The method of claim 38 wherein the semiconductor device includes a first component including the first bonding location and a second component including the second bonding location, the method further comprising the step of scanning eyepoints of each of the first component and the second component prior to each of the repeated inspecting steps.

40. The method of claim 38 wherein at least a portion of the repeating of the inspecting of the predetermined portions of the wire loops is conducted successively in a single pass conducted in the order in which the wire loops were wire bonded on the wire bonding machine, whereby the pattern recognition system obtains multiple images of each of the predetermined portions of the wire loops prior to moving to the next bonding location in the order in which the bonding locations were wire bonded.

41. The method of claim 38 wherein at least a portion of the repeating of the inspecting of the predetermined portions of the wire loops is conducted successively in multiple passes conducted in the order in which the bonding locations were wire bonded on the wire bonding machine, whereby the pattern recognition system obtains at least one image of each of the predetermined portions of the wire loops during each of the multiple passes.

42. The method of claim 38 further comprising the step of arithmetically deriving more accurate inspection data of the predetermined portions of the wire loops by utilizing the inspection data obtained from the repeated inspecting of the predetermined portions of the wire loops.

43. The method of claim 42 wherein the step of arithmetically deriving more accurate inspection data includes averaging the inspection data for each of the bonding locations from each of the repeated inspecting steps of the repeated inspecting.

44. The method of claim 35 wherein the step of inspecting includes scanning a first ball bond portion of at least a portion of the wire loops and comparing a scanned ball bond position of the scanned ball bond portion with a desired position of the first ball bond portion on the respective bonding location.

45. The method of claim 35 further comprising the step of (3) inspecting predetermined portions of wire loops on another semiconductor device.

46. The method of claim 45 wherein step (3) includes using the pattern recognition system of the wire bonding machine to scan a first bond portion of the wire loops on the another semiconductor device, step (3) being conducted by scanning the first bond portion of the wire loops successively and in order without inspecting any other portion of the wire loops.

47. A computer readable carrier including computer program instructions which cause a computer to implement a method of inspecting wire loops of a semiconductor device on a wire bonding machine, the method comprising the steps of:
(1) providing a semiconductor device including a plurality of wire loops, each of the wire loops providing electrical interconnection between a first bonding location of the semiconductor device and a second bonding location of the semiconductor device; and
(2) inspecting predetermined portions of the wire loops using a pattern recognition system of the wire bonding machine, the inspecting step being conducted by moving a portion of the pattern recognition system to scan the predetermined portions of the wire loops at the respective bonding locations in the order in which they were wire bonded on the wire bonding machine.

48. A wire bonding machine comprising:
a pattern recognition system for inspecting portions of wire loops previously bonded using the wire bonding machine, each of the wire loops providing electrical interconnection between a first bonding location of the semiconductor device and a second bonding location of the semiconductor device; and
a control system configured to control operation of the pattern recognition system to obtain inspection data related to predetermined portions of the wire loops, the control system being configured to move a portion of the pattern recognition system to scan the predetermined portions of the wire loops at respective bonding locations in the order in which they were wire bonded on the wire bonding machine.
